(19) United States
(12) Patent Application Publication
(10) Pub. No.: US 2026/0365686 A1
(43) Pub. Date: Nov. 19, 2026

(54) PLATINUM(II)-BASED ANTICANCER AGENTS, TARGETED CONJUGATES, AND METHOD OF SYNTHESIZING THEREOF

(71) Applicant: University of Massachusetts, Westborough, MA (US)

(72) Inventors: Maolin Guo et al., West Roxbury, MA (US); Yifei Dou, Ridgewood, NJ (US)

(21) Appl. No.: 19/123,356

(22) PCT Filed: Oct. 25, 2023
(86) PCT No.: PCT/US2023/077788

§ 371 Date: Apr. 22, 2025

Related U.S. Application Data
(60) Provisional application No. 63/380,863, filed on Oct. 25, 2022.

(51) Int. Cl. C07F 15/00; A61K 38/18; A61K 47/64

(52) U.S. Cl. CPC C07F 15/0093; A61K 38/1808; A61K 47/64

(57) ABSTRACT

Methods of synthesizing diamine platinum(II) complexes and their conjugates are disclosed.

### CROSS-REFERENCE TO RELATED APPLICATIONS

This application claims priority to U.S. provisional patent application No. 63/380,863, which was filed on Oct. 25, 2022, the disclosure of which is hereby incorporated by reference in their entireties.

### BACKGROUND OF THE INVENTION

Platinum anticancer drugs have been employed over decades for the single therapy or combination with radiotherapy of various solid tumors, such as, colorectal, breast, non-small cell lung and gastric cancers. There have been seven platinum (II)-based drugs approved in clinical use. However, the clinical application of the Pt(II) anticancer drugs is limited by low solubility, drug resistance, and dose-limiting side effects. The following dose-limiting side effects have been observed: nephrotoxicity for cisplatin, and heptaplatin (Miller et al., 2010; Ahn J. H et al., 2002); myelosuppression for carboplatin, nedaplatin, and lobaplatin (Shimada et al., 2013; Makovec 2019); neurotoxicity for oxaliplatin (Kelland, 2007); and gatrointestinal toxicity for oxaliplatin (Gebremedhn et al., 2018). Other common side effects for platinum (II) anticancer drugs include hepatotoxicity, ototoxicirty, alopecia, nausea and vomiting (Boztepe et al., 2021). Pt(IV)-based drugs have also been developed but none are approved for clinical usage so far.

Platinum anticancer drugs can have severe side effects because of their poor selectivity for cancerous tissue over normal tissue (Oun et al., 2018). Targeting and delivery of Platinum-based drugs specifically to cancer cells is an ideal approach to avoid the normal tissue damage and drug resistance.

Previously syntheses of Pt-based drugs include a targeting moiety tethered mostly to an amine or imine functional group on Pt(II) or a linker at one of the axial ligands of a Pt(IV) center. Such syntheses are multi-step chemical synthesis. In addition, Pt(IV)-based drugs are prodrugs, not the drug itself. Pt(IV) needs to be converted to Pt(II) (by light or reducing agents) for anticancer action. Despite a few clinical trials, no Pt(IV) drugs have been approved, due to efficacy or toxicity issues.

Accordingly, there is a need in the industry for new Pt(II)-based anti-cancer drugs, that is more easily synthesized and efficacious, with improved water solubility, reduced side-effects, and toxicity than prior art Pt-based drugs, and new targeted Pt(II)-based drugs.

### SUMMARY OF THE INVENTION

[Will complete once claims are finalized.]

### BRIEF DESCRIPTION OF THE DRAWINGS

Features, aspects, and advantages of the present invention will become better understood with reference to the following description, appended claims, and accompanying drawings where:

FIG. 1 is a scheme showing an approach for creating new Pt(II) complexes containing a free carboxyl group for specific conjugated Pt(II) complexes.

FIG. 2 is a representation of a Pt(II)-based complex comprising multicarboxylate ligands, where the Pt(II)-based complex is nitrilotriacetic acid (NTA)-platin.

FIG. 3 is a chart of the kinetics of the reaction of guanosine-5′-monophosphate (5′-GMP) with tricarboxylic acid (TCA)-platin in a molar ratio of 2:1 in deuterium oxide at 37 °C investigated using ¹H NMR (H8 signal); the downfield shift of the H-8 peak is assignable to the formation of the Pt-adduct.

FIG. 4 is a plot comparing the kinetics of 5′-GMP binding with cisplatin, and other platinum complexes in a ratio of 2:1 in deuterium oxide.

FIG. 5 are charts of cytotoxicity of cisplatin (2-128 μM) on Caco-2(a) and HepG2(b) cell lines.

FIG. 6 are charts of cytotoxicity of an embodiment of a platinum(II) complex (TCA-platin (2-1,024 μM) on Caco-2(e) and HepG2(f) cell lines at 4 days.

FIG. 7 are charts of dose-dependent cytotoxicity of platinum complexes against Caco-2 cell line.

FIG. 8 are charts of dose-dependent cytotoxicity of platinum complexes on HepG2 cell line.

FIG. 9 are charts of dose-dependent cytotoxicity of carboplatin, CIT-platin and CIT-platin-HER2 conjugates-on SKBR-3, MDA-MB-453, and MCF-7 cell lines.

FIG. 10 is a table showing the results of an assay employing 3-(4,5-dimethylthiazol-2-yl)-2,5-diphenyltetrazolium bromide (MTT) to determine the IC₅₀ against two human cancer cell lines of 10 new platinum(II) based complexes. The complexes were tested for their in vitro anticancer effects using cisplatin and carboplatin as positive controls. All the new platinum complexes except NTA-platin showed better anticancer activities than carboplatin in both the cell lines with the ones with the best activity highlighted in the red frames.

FIG. 11 shows images of morphological changes of HepG2 cells treated with cisplatin or TCA-platin, where inset A, is a control; inset B is 10 μM cisplatin; inset C is 10 μM TCA-platin; inset D is 45 μM TCA-cisplatin, and the blue fluorescence indicates H033342, and the red fluorescence represents PI accumulation, and the red arrows show apoptotic nuclear.

FIG. 12 shows representative confocal images of HepG2 cells detected with double staining of HO/PI. Cells were treated with vehicle, 10 μM cisplatin, 10 μM TCA-platin, or 45 μM TCA-platin. The blue fluorescence indicates H033342 stain (Hoechst stain), and the red fluorescence represents PI accumulation. All scale bar set for 10 m.

FIG. 13 shows morphological changes of SKBR-3 cells treated with carboplatin or CIT-platin. A, control; B, 70 μM carboplatin; C, 3 μM CIT-platin; D, 11 μM CIT-platin. The blue fluorescence indicates H033342, and the red fluorescence represents PI accumulation. The white arrow shows apoptotic nuclear. All scale bar set for 10 μm.

FIG. 14 shows representative confocal images of SKBR-3 cells detected with double staining of HO/PI. Cells were treated with vehicle, 70 μM carboplatin, 3 μM CIT-platin, or 11 μM CIT-platin. The blue fluorescence indicates H033342, and the red fluorescence represents PI accumulation. All scale bar set for 10 m.

FIG. 15 is a scheme of a one-step synthesis of TCA-platin-HER2 conjugates.

FIG. 16 is an HPLC profile of TCA-platin-HER2 Peptide conjugates.

FIG. 17 is an HR-MS spectrum of the TCA-platin-HER2 Peptide conjugates.

### DETAILED DESCRIPTION

The disclosure relates to platinum(II)-based anticancer agents and targeted conjugates, and more particularly to an improved method of synthesizing said platinum(II)-based anticancer agents. In one example, the Pt(II)-based complexes of the disclosure comprise multicarboxylate/hydroxyl ligands that enhance the water solubility of the complexes. The increased water solubility of the Pt(II)-based complexes of the disclosure helps improve their efficacy as well as potentially reducing side-effects.

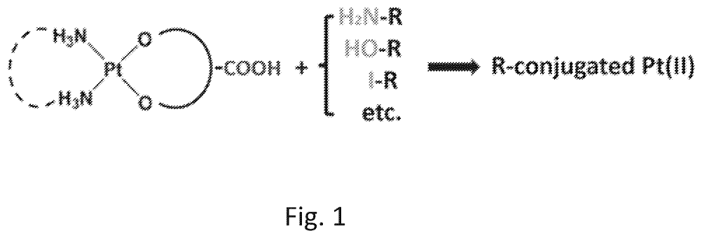

The Pt(II) complexes of the disclosure contain a carboxyl group(—COOH, also referenced herein as a “free carboxyl group”) that can be directly conjugated with a targeting peptide or antibody that can be recognized by aberrantly expressed cell surface receptors, for selective delivery of the platinum anticancer drug to cancer cells. The free carboxyl group can be used for conjugating to various functional groups for specific usage, such as tumor-specific drug delivery. (FIG. 1).

Previously made Pt-based drug conjugates largely involve the targeting moiety being tethered mostly to an amine or imine functional group on Pt(II) or a linker at one of the axial ligands of a Pt(IV) center, making it a multi-step chemical synthesis. In addition, Pt(IV)-based drugs require an activation step that reduces the Pt(IV) center to Pt(II) (e.g., by light or reducing agents). In contrast, the disclosure relates to a conjugation based on Pt(II) drugs, which can directly act on targets without the need of any activation step.

The disclosure relates to a diamine platinum(II) complex, comprising:
(1) two amino ligands or substituted amino ligands; and
(2) a polycarboxyl ligand (e.g., di- or tri-carboxyl ligand optionally substituted with a hydroxyl group);
   wherein at least one of the carboxyl groups of the polycarboxyl ligand is a free carboxyl group that can be conjugated to a functional moiety. Examples of a suitable “functional moiety” includes a peptide, an antibody, a nanoparticle, or another functional moiety, as described herein.

The Pt(II) complexes can be of Formula (I) or (II):

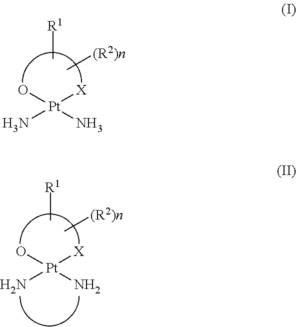

or a pharmaceutically acceptable salt (e.g., carboxylate salts) thereof, wherein:
R¹ is a hydroxyl group (OH), a carboxyl group (COOH) or a carboxyalkyl group (e.g., a C₁-C₄—COOH group);
n is 0, 1 or 2;
R² is a hydroxyl group, a carboxyl group (COOH) or a carboxyalkyl group (e.g., a C₁-C₄—COOH group) or two R² groups, together with the atom to which they are attached, can form a double bond (e.g., a double bond optionally substituted with R¹, such as the group ═CHCOOH) or an acyl (CO) group; or
two R² groups, together with the atoms to which they are attached, can form a double bond, a cycloalkyl group or an aryl group, provided that when two R² groups, together with the atoms to which they are attached for an a cycloalkyl group or an aryl group, R¹ can be located on the cycloalkyl group or the aryl group, respectively;
X is O or NR², wherein R² is H or a carboxyalkyl group; and
the ring formed by:

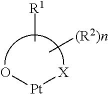

can be a five-, six-, seven- or eight-membered ring and
the ring formed by:

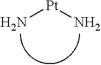

can be a five-, six-, seven- or eight-membered ring that can be fused with another five- or six-membered cycloalkyl or aryl ring.

In one example, R¹ is OH or COOH. Alternatively, or in addition, n is 1 and R² is OH or COOH.

In another example, R¹ is OH or COOH, n is 3, one R² is OH or COOH and the other two R² groups form an acyl group. In this example, the group of the formula:

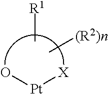

in Formula (I) or (II) alike) can be a group of the formula:

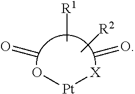

An example of the group:

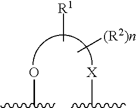

on compounds of the Formula (I) and (II) includes a group of the formula:

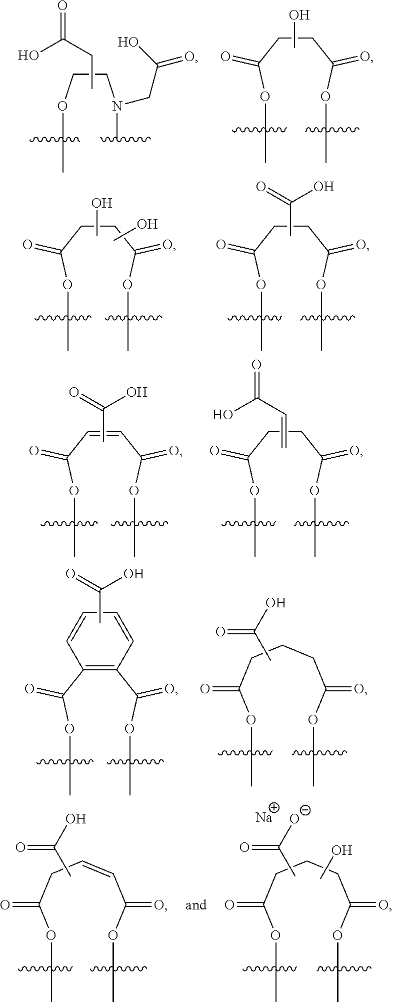

including diastereomers thereof. Examples of the foregoing groups include:

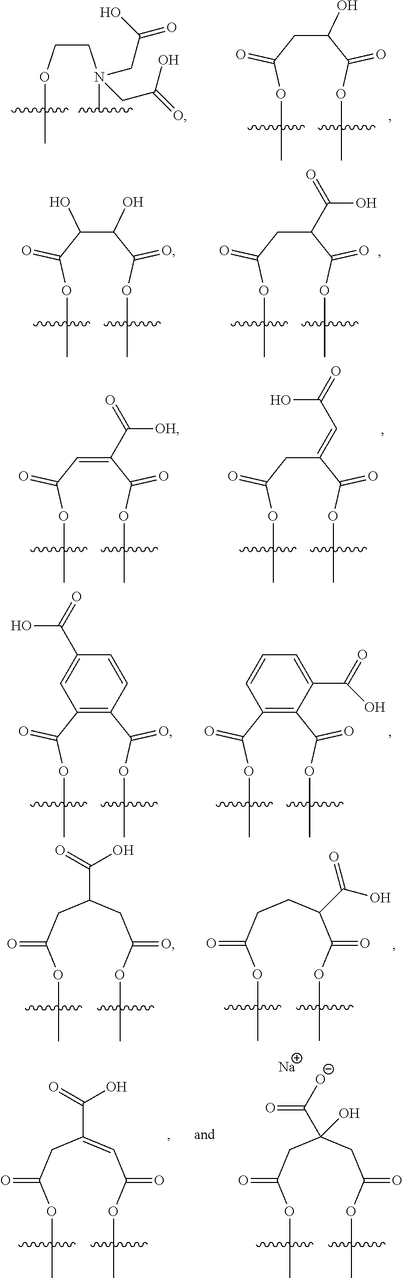

and diastereomers thereof, such as:

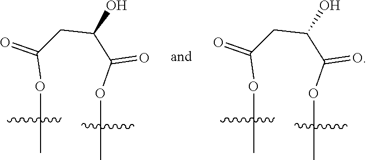 [SMILES: *OC(=O)C[C@@H](O)C(=O)O* ; *OC(=O)C[C@H](O)C(=O)O*]

Examples of the ring formed by the group:

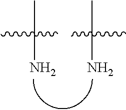

can be:

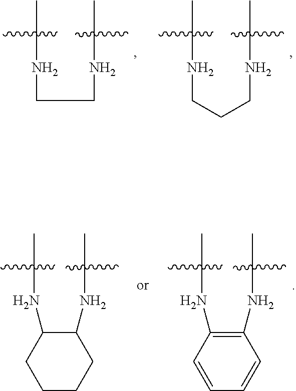 [SMILES: *NCCN* ; *NCCCN* ; *NC1CCCCC1N* ; *Nc1ccccc1N*]

Examples of the Pt(II) contemplated herein include complexes selected from:

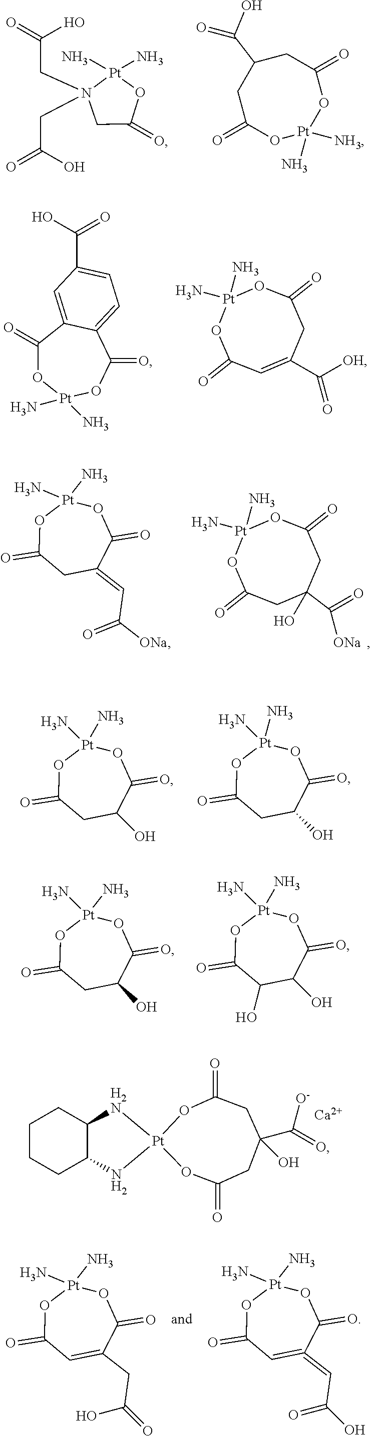

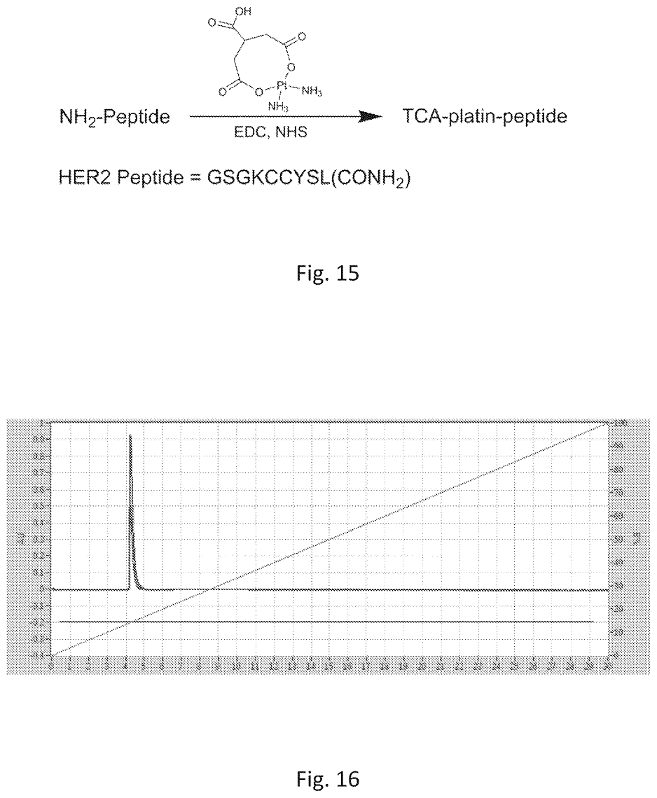

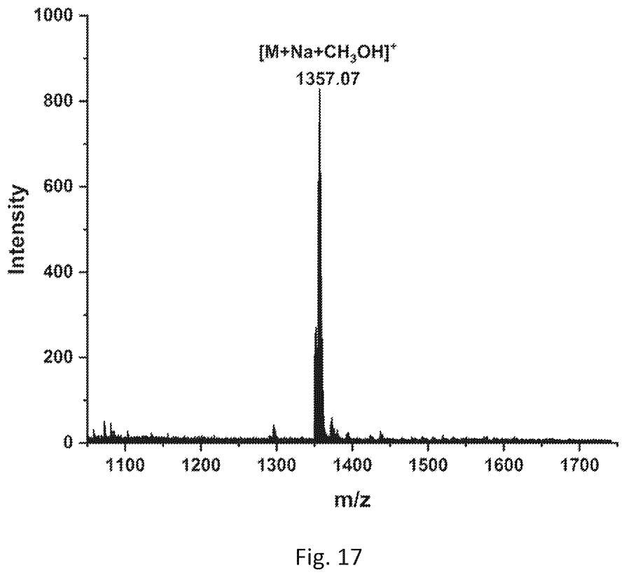

In embodiments, the free carboxyl moiety (COOH) in the Pt(II) complexes of the disclosure enables a direct one-step conjugation with a peptide, an antibody, a nanoparticle, or another functional moiety. The free carboxyl group can be used for conjugation to various functional groups (e.g., amino groups NH₂R′, hydroxyl groups HOR′, or organoiodine IR′) for specific usage, such as for tumor-specific drug delivery. For example, a HER2 peptide (e.g., a peptide with the amino acid sequence GSGKCCYSL-C(O)NH₂) can be conjugated to the Pt(II) complexes of the disclosure to form a Pt(II) complex for targeting HER2 positive cancer tissues. An example of such a conjugate is shown below and in FIG. 15:

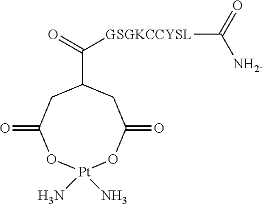

The disclosure also relates to methods of synthesizing Pt(II) complexes of Formula (I) and (II). In one example, the method of synthesizing a Pt(II) complex of Formula (I) and (II) comprises:
contacting a starting Pt(II) complex of the general formula (L)₂Pt(amine)₂ with a polycarboxyl compound in the presences of a silver, mercury, or lead salt to give a Pt(II) complex product (e.g., of Formula (I) or (II);
wherein:
each L independently represents a leaving group; and
the Pt(II) complex product comprises at least one free carboxyl group (e.g., one that is uncoordinated). The leaving group, L, can be any suitable leaving group. For example, each L can be a chloride. Other leaving groups include bromide, iodide and thiocyanate. Each amine group in the starting Pt(JJ) complex of the general formula (L)₂Pt(amine)₂ can independently be any suitable amine, such as NH₃, NH₂R³ (wherein R³ is alkyl), an amine of the formula NH₃-alkyl-NH₃, such as:

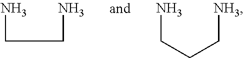

an amine of the formula NH₃-cycloalkyl-NH₃, such as:

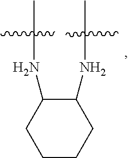 [SMILES: *NC1CCCCC1N*]

or an amine of the formula NH₃-aryl-NH₃, such as

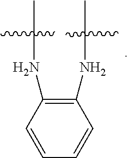 [SMILES: *Nc1ccccc1N*]

For example, compounds of the Formula (I) can be synthesized as follows:

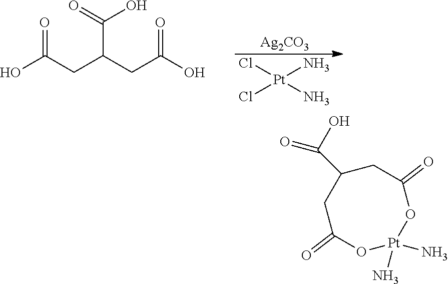

where the tricarboxylic acid is reacted with the dichloro diamino Pt(II) complex to form the diamino product where two of the starting material's carboxyl groups are coordinated to the Pt(II) center and the third carboxyl group is a “free” or “uncoordinated” carboxyl. This process can be carried out in the in the presence of the silver salt Ag₂CO₃ or any other suitable reagent. Other silver salts that can be used in the methods described herein include AgNO₃ and the like. The method can be carried out in the presence of a base. The base can be NaOH, KOH, LiOH, Ca(OH)₂, or Mg(OH)₂. The reaction can be carried out in the presence of a salt. The salt can be NaCl, KCl, LiCl, CaCl₂), or MgCl₂

Examples of suitable polycarboxyl compounds that can be used to prepare compounds of Formula (I) or (II) of the disclosure include:

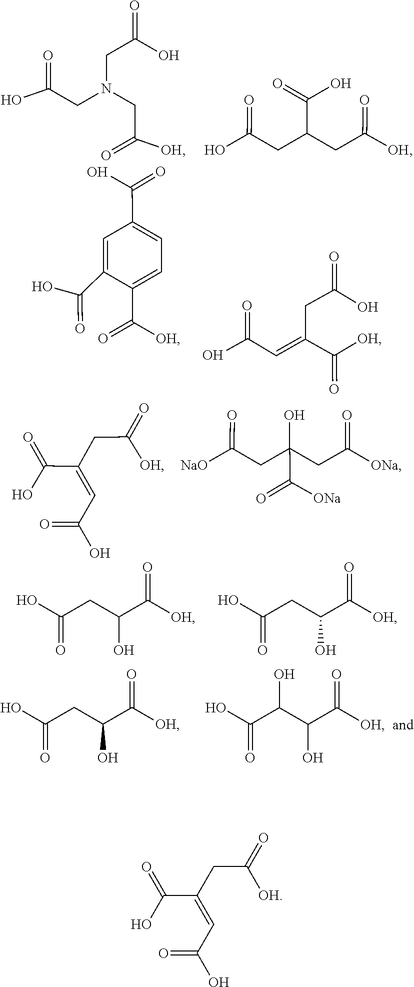

The Pt(II) complex product can be further conjugated via the at least one free carboxyl group with a targeting moiety, such as a peptide, an antibody, a nanoparticle or another functional moiety. As discussed herein, the peptide can be a HER-2 peptide, such as a HER-2 peptide comprising the sequence GSGKCCYSL, KTIYYLGYYNPNEYRY, RSLWSDFYASASRGP, or D-form peptide D-(RNWELRLK), etc. In embodiments, the targeting moiety is selected for delivery of the Pt(II) drug to cancer cells. In other embodiments, the targeting moiety can include a cancer-targeting moiety, such as a folate ligand, a peptide, a nanoparticle, or an antibody configured to specifically deliver the Pt(II) complexes into cancer cells. The targeting moiety (e.g., folate ligand, peptide, nanoparticle, or antibody) can be linked directly to the Pt(II)-based complexes of the disclosure directly via the uncoordinated carboxyl group or the targeting moiety (e.g., folate ligand, peptide, nanoparticle, or antibody) can be linked to the Pt(II)-based complexes of the disclosure via a linker that is conjugated to the uncoordinated carboxyl group via an amide bond or an ester bond or any suitable bond.

In embodiments, the Pt(II)-based complex has enhanced water-solubility. For example, the Pt(II)-based complexes of the disclosure can have a 1.5-, 2-, 3—, 5-, 10-, 15-, 20-, 30-, 50-, 80- or a 100-fold greater water solubility than cisplatin. In embodiments, the Pt(II)-based complex has reduced side-effects over other drugs such as cisplatin.

It would be appreciated by those skilled in the art that various changes and modifications can be made to the illustrated embodiments without departing from the present invention. All such modifications and changes are intended to be within the scope of the present invention except as limited by the scope of the appended claims.

### CERTAIN DEFINITIONS

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

As used herein “aromatic” refers to an unsaturated cyclic molecule having 4n+2 π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

“Targeting moiety” refers to a functional group (e.g., a peptide, an antibody, a nanoparticle or other molecules or macromolecules) that bind to specific receptors located on the surface of the tumoral cells.

### EXAMPLES

### Example 1: Synthesis of NTA-Platin

Silver carbonate (1.6914 g, 6.13 mmol) and nitrilotriacetic acid (1.1752 g, 6.15 mmol) were dissolved in 40 ml deionized water at room temperature. The reaction mixture was stirred at room temperature for 30 min. The mixture was filtered to obtain a brownish grey residue which was dried in vacuum overnight and was then ground to make a grey powder (2.0566 g) which was subsequently mixed with cisplatin (1.5103 g, 5.03 mmol) in 50 ml deionized water at room temperature. The reaction solution was stirred at room temperature in the dark overnight. The solution was filtered to obtain an offwhite filter liquor to which activated carbon was added and then the mixture was left to stand for 15 min. The activated carbon was removed by filtration to obtain a light-yellow clear solution. Then the solution was rotary evaporated to obtain a 10 ml yellow solution which was dried under vacuum for 4 days. The product was obtained after grinding with the following yield: 1.1950 g (54.5%). The formula is C6H13N3O6Pt·H2O. ESI-MS: m/z calcd for C6H13N3O6Pt [M+H]+ is 419.05, found 418.94. ¹³C NMR (101 MHz, Deuterium Oxide) δ 185.34, 171.91, 64.22, 62.12. (Scheme 1)

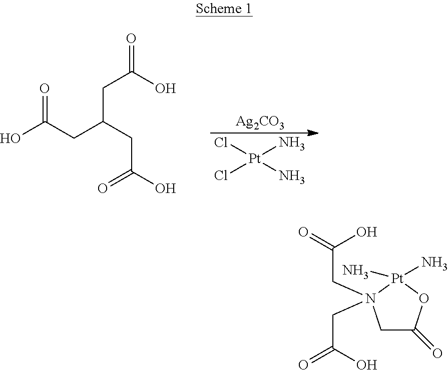

### Example 2: Synthesis of TCA-Platin

Silver carbonate (1.6928 g, 6.14 mmol) and tricarballylic acid (1.0743 g, 6.10 mmol) were dissolved in 40 ml deionized water at room temperature. The reaction mixture was stirred at room temperature for 30 min. The mixture was filtered to obtain a light grey residue which was dried in vacuum overnight. The residue was ground to obtain a grey powder (2.1421 g) and mixed with cisplatin (1.7460 g, 5.82 mmol) in 50 ml deionized water at room temperature. The reaction solution was stirred at room temperature in the dark overnight. The solution was filtered to obtain a light-yellow filter liquor to which activated carbon was added. This mixture was left to stand for 15 min. The activated carbon was removed by filtration to obtain a light-yellow clear solution which was subsequently evaporated on a rotary evaporator and dried under vacuum for 4 days. The product was ground to obtain a fine powder. Yield: 0.306 g (13.1%). ESI-MS: m/z calcd for C6H12N2O6Pt, [M+H]⁺ 404.03, found 404.01. ¹H NMR (400 MHz, Deuterium Oxide) δ 2.77 (p, J=7.6 Hz, 1H), 2.34 (dd, J=15.1, 7.7 Hz, 2H), 2.08 (dd, J=15.1, 7.5 Hz, 2H). ¹³C NMR (101 MHz, D2O) δ 181.30, 168.59, 43.33, 40.36. (Scheme 2)

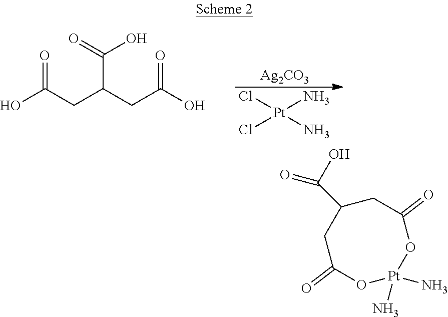

### Example 3: Synthesis of TMA-Platin

Silver carbonate (0.5534 g, 2.01 mmol) and trimellitic acid (0.4258 g, 2.00 mmol) were dissolved in 50 ml deionized water at room temperature. The reaction mixture was stirred at room temperature for 1 hour. The mixture was filtered to obtain a light yellow residue which was dried in vacuum overnight. The residue was ground to obtain a light-yellow powder (0.6937 g) and mixed with cisplatin (0.3911 g, 1.30 mmol) in 50 ml deionized water at room temperature. The reaction solution was stirred at room temperature in the dark overnight, and then filtered to obtain a light blue clear filter liquor. This light blue solution was evaporated on a rotary evaporator and dried under vacuum for 4 days. The blue powder was obtained after grind. Yield: 0.0712 g (12.53%). ¹H NMR (400 MHz, Deuterium Oxide) δ 8.28 (s, 1H), 8.08 (d, J=9.0 Hz, 1H), 7.65 (d, J=8.0 Hz, 1H). ESI-MS: m/z calcd for C9H10N2O6Pt, [M+H]⁺ 438.02, found 438.00. (Scheme 3)

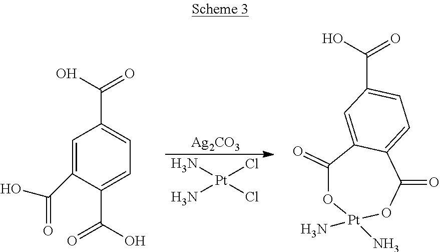

### Example 4: Synthesis of TAA-Platin

Silver carbonate (0.8624 g, 3.12 mmol) and trans-aconitic acid (1.3939 g, 8.0 mmol) were dissolved in 30 ml deionized water at room temperature. The reaction mixture was stirred at room temperature for 1 hour and then filtered to obtain a light grey residue which was dried under vacuum overnight. The residue was then ground to obtain a grey powder (0.7714 g) and mixed with cisplatin (0.4764 g, 1.59 mmol) in 50 ml deionized water at room temperature. This solution was then stirred at room temperature in the dark overnight. The solution was then filtered to obtain a light yellow clear filter liquor which was subsequently evaporated on a rotary evaporator and dried under vacuum for 4 days. The brownish red powder was obtained after grind. Yield: 0.1198 g(18.78%). 1H NMR (400 MHz, Deuterium Oxide) δ 6.67 (s, 1H), 3.48 (s, 2H). ESI-MS: m/z calcd for C6H10N2O6Pt, [M+H]⁺ 402.02, found 402.01. (Scheme 4)

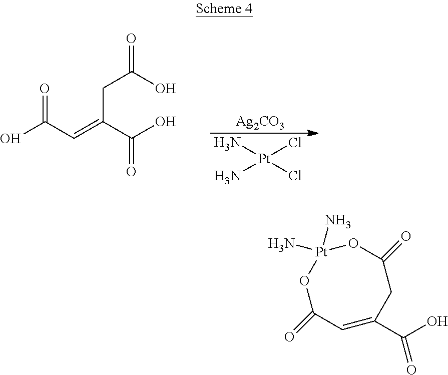

### Example 5: Synthesis of CAA-Platin

Cis-Aconitic acid (0.5000 g, 2.87 mmol) was dissolved in 30 ml deionized water at room temperature. 4.24 ml of 1M NaOH was added into solution to obtain cis-aconitic acid sodium salt(pH~8.0). Separately, silver nitrate (0.7798 g, 4.59 mmol) was dissolved in 10 ml deionized water. The molar ratio of acid/sodium:silver was 1:3.2. The silver nitrate solution was then added into the salt solution, immediately forming a light brown precipitate. The mixture was filtered to obtain a brown solid precipitate that was dried overnight and ground into a fine powder. The cis-aconitic acid-silver powder (0.5830 g, 1.18 mmol), cisplatin (0.3530 g, 1.18 mmol), and sodium chloride (0.0695 g, 1.18 mmol) were dissolved in 40 ml deionized water. This reaction mixture was stirred at room temperature in the dark overnight and subsequently filtered to obtain a dark green clear filter liquor. This green filter liquor was evaporated on a rotary evaporator and dried under vacuum for 4 days. The product was obtained after grind. Yield: 0.2162 g (45.68%). ESI-MS: m/z calcd for C6H9N2O6Pt, [M−H]⁻ 400.02, found 400.13. 1H NMR (400 MHz, Deuterium Oxide) δ 5.87 (s, 1H), 3.09 (d, 2H). (Scheme 5)

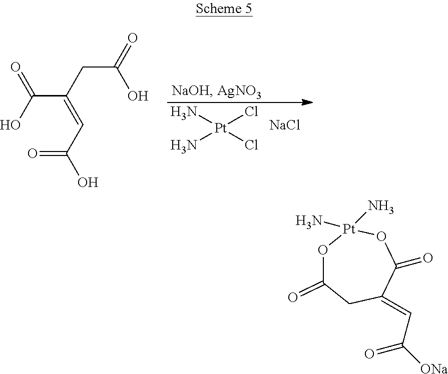

### Example 6: Synthesis of CIT-Platin

Citric acid trisodium (1.2887 g, 0.005 mol) was dissolved in 22 ml deionized water and, separately, silver nitrite (3.1444 g, 0.0185 mol) was dissolved in 23 ml deionized water at room temperature. The two solutions were mixed together, immediately forming a white precipitate. This mixture was filtered to obtain a white solid which was dried overnight. This white solid mixture was ground and weighed. The citric acid-silver powder (1.0616 g, 2.1 mmol), cisplatin (0.6299 g, 2.1 mmol), and sodium chloride (0.1231 g, 2.1 mmol) were dissolved in 50 ml deionized water. The reaction mixture was stirred at room temperature in the dark overnight and subsequently filtered to obtain a light-yellow clear filter liquor. This yellow solution was evaporated on a rotary evaporator and dried under vacuum for 4 days. The product was obtained after grind. Yield: 0.4407 g(47.56%). 1H NMR (400 MHz, Deuterium Oxide) δ 2.60 (d, J=15.1 Hz, 2H), 2.50 (d, J=15.2 Hz, 2H). ESI-MS: m/z calcd for C6H12N2O7Pt, [M+H]⁺ 420.03, found 420.06.(Scheme 6)

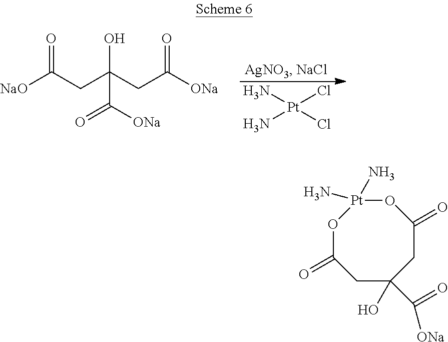

### Example 7: Synthesis of (DL-)MLA-Platin

Malic acid (DL-, 0.3012 g, 2.25 mmol) was dissolved in 20 ml deionized water at room temperature. 4.5 ml of 1M NaOH was added to this solution to obtain malic acid sodium salt(pH-8.0). Separately, silver nitrate (0.8310 g, 4.89 mmol) was dissolved in 10 ml deionized water. The molar ratio of acid/sodium:silver was 1:2.17, the silver nitrate solution was added into the salt solution, immediately forming a light brown precipitate. This mixture was filtered to obtain a brown solid that was dried overnight and ground into a fine powder. The malic acid-silver powder (0.6293 g, 1.8 mmol), and cisplatin (0.5323 g, 1.77 mmol) were dissolved in 40 ml deionized water. This reaction mixture was stirred at room temperature in the dark overnight and then filtered to obtain a yellow-green clear filter liquor. This yellow-green solution was evaporated on a rotary evaporator and dried under vacuum for 4 days. The product was obtained after grind. Yield: 0.3953 g(61.58%). ESI-MS: m/z calcd for C4H10N2O5Pt, [M+H]⁺ 362.02, found 362.01. (Scheme 7).

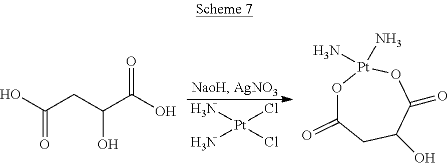

### Example 8: Synthesis of D-MLA-Platin

D-Malic acid (0.2030 g, 1.51 mmol) was dissolved in 20 ml deionized water at room temperature. 3.0 ml of 1M NaOH was added to the D-malic acid solution to obtain D-malic acid sodium salt(pH~8.0). Separately, silver nitrate (0.5437 g, 3.2 mmol) was dissolved in 20 ml deionized water. The molar ratio of Acid/Sodium:Silver was 1:2.1. Then the silver nitrate solution was added into the salt solution, immediately forming a white precipitate. This mixture was filtered, and the white solid was obtained and allowed to dry overnight and ground into a fine powder. The D-malic acid-silver powder (0.4219 g, 1.21 mmol), and cisplatin (0.3089 g, 1.03 mmol) were dissolved in 50 ml deionized water. The reaction mixture was stirred at room temperature in the dark for 2 days and then filtered to obtain a clear filter liquor. This solution was evaporated on a rotary evaporator and dried under vacuum for 4 days. The product was obtained after grind. Yield: 0.2504 g(67.28%). ESI-MS: m/z calcd for C4H10N2O5Pt, [M+H]⁺ 362.02, found 362.01. (Scheme 8)

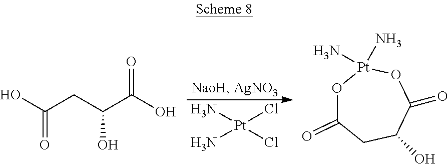

### Example 9: Synthesis of L-MLA-Platin

L-Malic acid (0.2047 g, 1.53 mmol) was dissolved in 20 ml deionized water at room temperature. 3.0 ml of 1M NaOH was added into solution to obtain L-malic acid sodium salt (pH~8.0). Separately, silver nitrate (0.5476 g, 3.32 mmol) was dissolved in 10 ml deionized water. The molar ratio of acid/sodium:silver was 1:2.1. The silver nitrate solution was added into the salt solution, immediately forming a white precipitate. This mixture was filtered to obtain a white solid that was dried overnight and ground into a fine powder. The L-malic acid-silver powder (0.2001 g, 0.57 mmol) and cisplatin (0.1587 g, 0.53 mmol) were dissolved in 50 ml deionized water. The reaction mixture was stirred at room temperature in the dark overnight and then filtered to obtain a clear filter liquor. This solution was evaporated on a rotary evaporator and dried under vacuum for 4 days. The product was obtained after grind. Yield: 0.1305 g (68.37%). ESI-MS: m/z calcd for C4H10N2O5Pt, [M+H]⁺ 362.02, found 362.00. (Scheme 9)

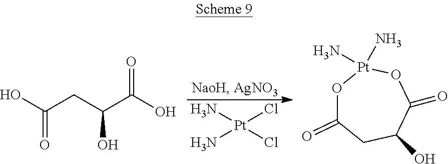

### Example 10: Synthesis of TTA-Platin

Tartaric acid (0.3171 g, 2.11 mmol) was dissolved in 20 ml deionized water at room temperature. 4.12 ml of 1M NaOH was added into solution to obtain tartaric acid sodium salt (pH~8.0). Separately, silver nitrate (0.7557 g, 4.45 mmol) was dissolved in 20 ml deionized water. The molar ratio of acid/sodium:silver was 1:2.16. The silver nitrate solution was added into the salt solution, immediately forming a white precipitate. The mixture was filtered to obtain a white solid that was subsequently dried overnight and ground into a fine powder. The tartaric acid-silver powder (0.6334 g, 1.7 mmol) and cisplatin (0.4988 g, 1.66 mmol) were dissolved in 40 ml deionized water. This reaction mixture was stirred at room temperature in the dark overnight and then filtered to obtain a clear filter liquor. This colorless solution was evaporated on a rotary evaporator and dried under vacuum for 4 days. The product was ground to obtain a fine powder. Yield: 0.1247 g (19.92%). ESI-MS: m/z calcd for C4H10N2O6Pt, [M−H]⁻ 376.02, found 376.15. ¹H NMR (400 MHz, Deuterium Oxide) δ 4.44 (d, J=2.4 Hz, 1H), 4.40 (d, J=2.3 Hz, 1H). ¹³C NMR (101 MHz, Deuterium Oxide) δ 175.41, 72.38. (Scheme 10)

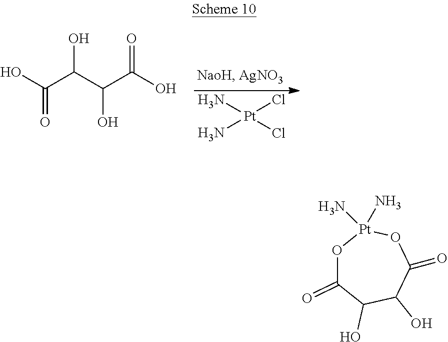

### Example 11: Synthesis of CIT-DACH-Platin

Two approaches were used to synthesis CIT-DACH-Platin. In Approach 1, oxaliplatin and calcium citrate (2 mM each) were each dissolved in deionized water at room temperature. The mixture was stirred for 8 h, forming a participate which was filtered out. The remaining solution was dried to produce the product CIT-DACH-Platin. In Approach 2, K₂PtCl₄ was dissolved in water and R,R-DACH (1 equiv.) was added and the solution was stirred for 1 hr. The resultant crystalline solid cis-[Pt(R,R-DACH)Cl₂] was filtered, and washed with ice-cold water and ethanol, and subsequently dried under vacuum. Cis-[Pt(R,R-DACH)Cl₂] was dissolved in water at room temperature and an aqueous solution of AgNO₃ (2 equiv.) was added and stirred in the dark overnight. A white precipitate was formed and removed by filtration. Then sodium citrate (1 equiv) was added to the filtrate and the solution was stirred for 2 d. The solvent was removed by freeze-drying in order to collect the product CIT-DACH-Platin. ESI-MS: m/z calcd for C12H19N2O7Pt, [M+H]⁺ 499.38, found 500.09; [2M+13H₂O+2H]²⁺ 615.50, found 615.56. (Scheme 11)

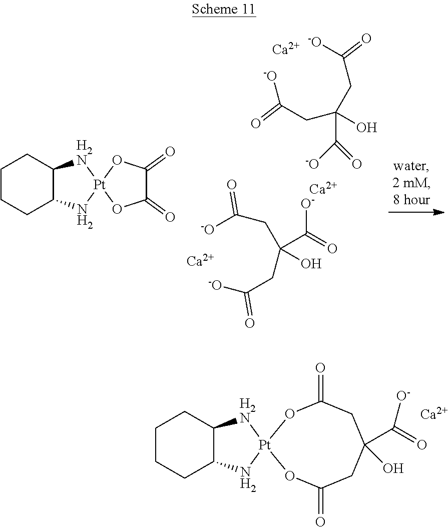

### Example 12: Synthesis of HER2-Conjugate Complexes

### Synthesis of TCA-Platin-HER2 Complex

TCA-platin (9.9 mg, 0.0246 mmol), 1-Ethyl-3-(3-dimethylaminopropyl)carbodiimide (EDC) (70.5 mg, 0.3697 mmol) and N-Hydroxysuccinimide (NHS) (19.2 mg, 0.1668 mmol) were dissolved in 8 ml of 0.05 M SPB buffer, pH 7.0, with stirring for 2 h at room temperature. The carboxyl group of the TCA-cisplatin was activated by EDC and NHS at a TCA-COOH/EDC/NHS=1:15:6.7 molar ratio. Then the HER2-peptide (18.9 mg, 0.0206 mmol) was added to the mixture at TCA-COOH/Peptide-NH2=1:0.8 molar ratio and continuously stirred for 1 h at room temperature. The mixture was purified by semi preparative reverse-phase HPLC. ESI-MS: m/z calcd for [M+Na+CH₃OH]⁺ is 1357.32, found 1357.07. (Scheme 12)

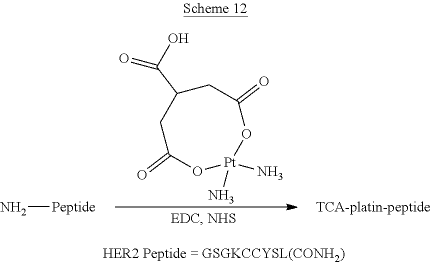

### Synthesis of CIT-Platin-HER2 Complex

CIT-platin (24.4 mg, 0.0553 mmol), 1-Ethyl-3-(3-dimethylaminopropyl)carbodiimide (EDC) (159 mg, 0.8295 mmol) and N-Hydroxysuccinimide (NHS) (42.6 mg, 0.3705 mmol) were dissolved in 15 ml of deionized water, with stirring for 2 h at room temperature. The carboxyl group of the CIT-platin was activated by EDC and NHS at a TCA-COOH/EDC/NHS=1:15:6.7 molar ratio. Then the Her2-peptide (38 mg, 0.0414 mmol) was added to the mixture at CIT-COOH/Peptide-NH2=1.25:1 molar ratio and continuously stirred for overnight at room temperature. The mixture was purified by semi-preparative reverse-phase HPLC (FIG. 15) and lyophilized to get the final product, Yield:70.31%. ESI-MS: m/z calcd for [M+10H₂O+Na+H]²⁺ is 761.14, found 761.28, calcd for [M+22H2O+3H]3+ is 572.43, found 572.14, and calcd for [M+9H₂O+2Na+2H]⁴⁺ is 382.07, found 382.03. (Scheme 13)

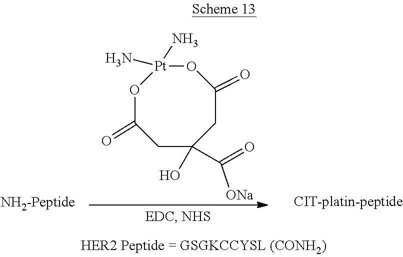

### Synthesis of CAA-Platin-HER2 Complex

CAA-platin (36.5 mg, 0.086 mmol), 1-Ethyl-3-(3-dimethylaminopropyl)carbodiimide (EDC) (244.9 mg, 1.278 mmol) and N-Hydroxysuccinimide (NHS) (69.3 mg, 0.602 mmol) were dissolved in 1.4 ml di-water with stirring for 2 h at room temperature. The carboxyl group of the CAA-cisplatin was activated by EDC and NHS at a TCA-COOH/EDC/NHS=1:15:6.9 molar ratio. Then the Her2-peptide (40 mg, 0.044 mmol) was added to the mixture at TCA-COOH/Peptide-NH2=2:1 molar ratio and continuously stirred for 1.5 h at room temperature. The mixture was purified by semi preparative reverse-phase HPLC. ESI-MS: m/z calcd for [M+H₂O+H]⁺ is 1319.16, found 1319.47. (Scheme 14)

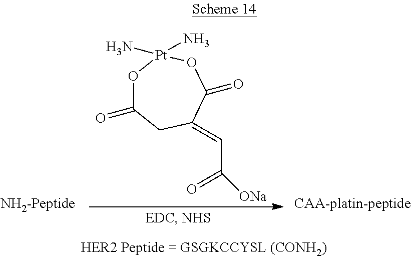

### Example 13: Solubility of the Platinum(II) Complexes

Each of the novel platinum complexes were dissolved in deionized water or PBS (phosphate buffer saline, pH 7.4) to obtain a supersaturated solution at room temperature. The supersaturated solution was filtered via a known weight 0.22 m syringe. Then the syringe filter was dried and weighed. After calculating, the solubility of each compound was obtained and listed below:

TABLE 2
Solubility of cisplatin and the new platinum complexes at 20 °C

| | | | | | | | | | DL- | D- | L- | CIT- |
|---|---|---|---|---|---|---|---|---|---|---|---|---|
| | | NTA- | TCA- | TAA- | CAA- | TMA- | CIT- | TTA- | MLA- | MLA- | MLA- | DACH- |
| Complex | Cisplatin | platin | platin | platin | platin | platin | platin | platin | platin | platin | platin | platin |
| Water | 1.14 | 35 | 2.3 | 1.92 | 62 | <1 | nt | nt | nt | nt | nt | nt |
| (mg/ml) | | | 3 | | | | | | | | | |
| PBS | 1.41 | 50 | 4.29 | 18 | 150 | 2.5 | 16.25 | 3.7 | 19.17 | 1.5 | 0.8 | nt |
| (mg/ml) | | | | | | | | | | | | |

nt = not tested

As summarized in Table. 2, most of the platinum(II)-based complexes have improved solubility compared to cisplatin in both pure water and PBS by different orders of magnitude. The platinum complexes showed better solubility in PBS than in water. CAA-platin complex showed the most significant increase in solubility—improved by 106-fold compared to that of cisplatin in PBS. The NTA-platin showed 35-fold increase compared to cisplatin in PBS. TAA-platin, CIT-platin and DL-MLA-platin increased 12-fold, 11-fold and 13-fold, respectively, compared to cisplatin in PBS. The solubility of DL-MLA-platin increased significantly compared to that of cisplatin (13-fold), however, that of D-MLA-platin or L-MLA-platin showed little increase compared to that of cisplatin in PBS.

### Example 14: Biological Activities

### DNA-Binding Activity

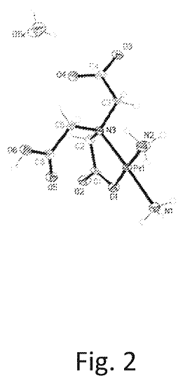

The DNA-binding reactivities of the new platinum-based complexes were studied by examining the kinetics of binding of each of the platinum complexes with guanosine-5′-monophosphate(5′-GMP) as was monitored by ¹H NMR spectroscopy. The platinum-DNA adduct formation was analyzed by integration of the H8 peak of 5′-GMP or the Pt-adducts and was compared to that of cisplatin. Cisplatin, carboplatin, NTA-platin, TCA-platin, TMA-platin, TAA-platin, CAA-platin, CIT-platin, DL-MLA-platin, TTA-platin, L-MLA-platin or D-MLA-platin were dissolved in 400 μl D₂O (0.01 mmol each) and 0.02 mmol of guanosine-5′-monophosphate free acid was dissolved in 400 μl SPB buffer(D₂O solvent) pH~7.0. Then 5′-GMP solution was added to each of the platinum complex solution to initiate the reaction. ¹H NMR were measured at 295 K over a period of several days until the reaction was almost complete (FIG. 2 as an example Kinetics of the reaction of 5′-GMP with Cisplatin in a molar ratio of 2:1 in deuterium oxide at 37 °C. The H-8 (8.0-8.5 ppm) and H-2 (5.5-6.0 ppm) 1H NMR peaks are shown. The downfield shift of the H-8 peak is assignable to the formation of the Pt-adducts.).

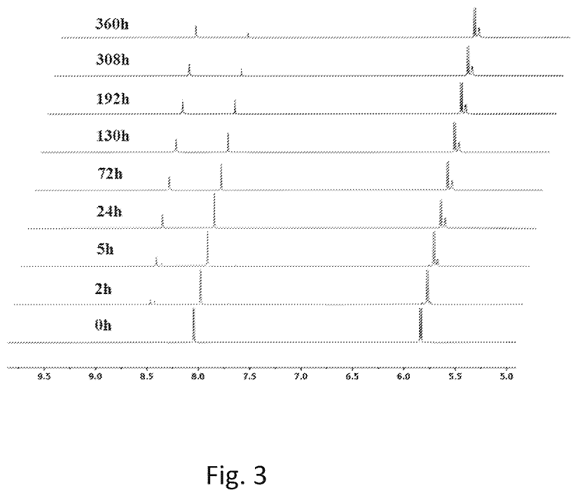

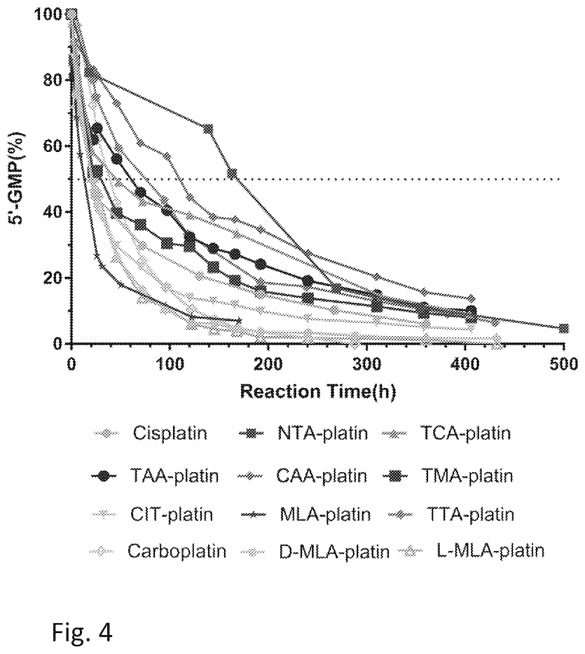

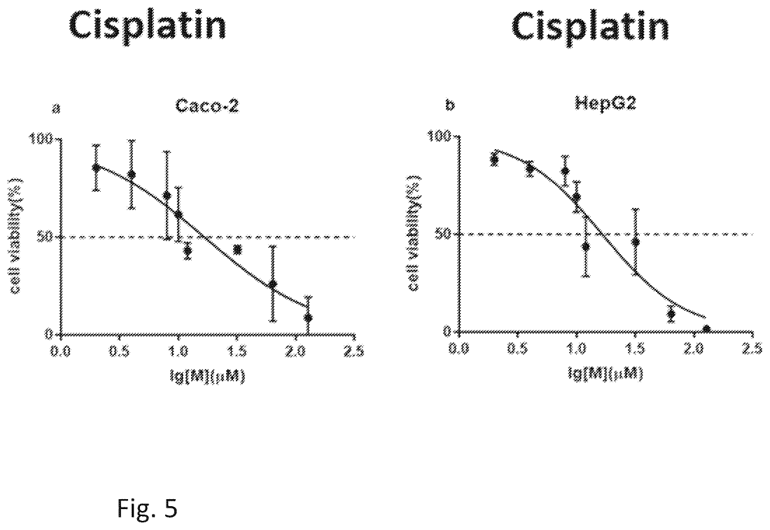

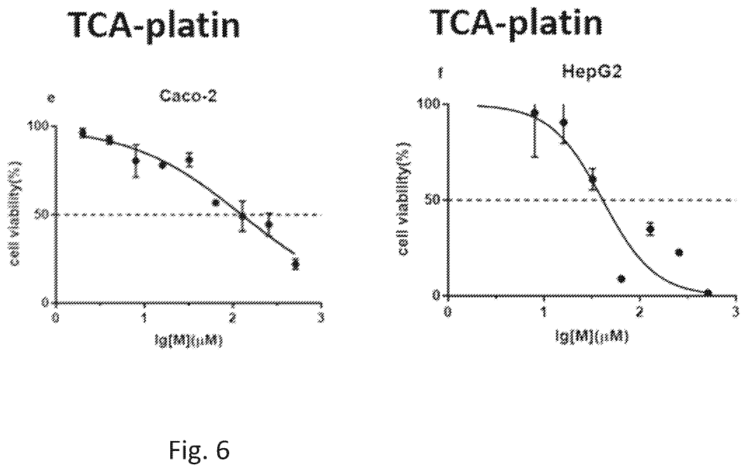

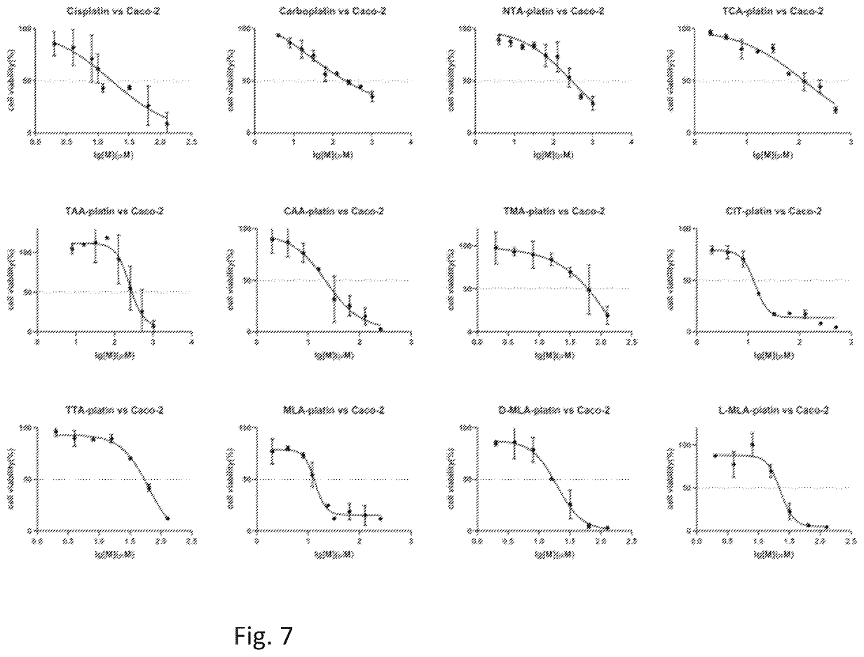

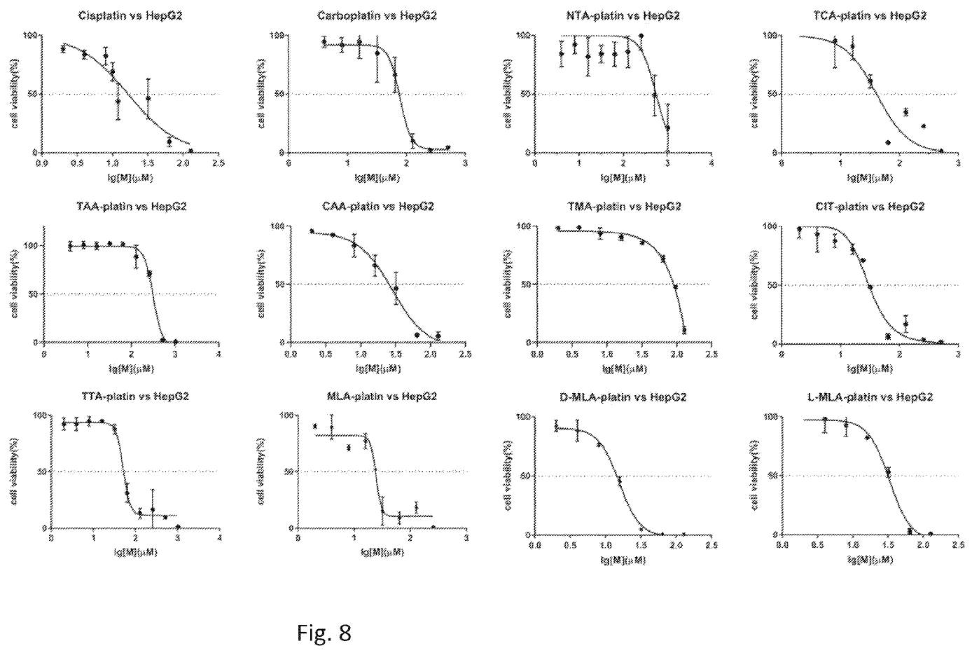

From the integration of the H8 peak data, the half-time (t_{1/2}) for 5′-GMP depletion with different platinum complexes was observed as follows (FIG. 3; Kinetic of 5′-GMP with cisplatin and the platinum complexes in a ratio of 2:1 in deuterium oxide.): cisplatin: t_{1/2}=22 h, carboplatin: t_{1/2}=39 h, NTA-platin: t_{1/2}=168 h, TCA-platin: t_{1/2}=44 h, TMA-platin: t_{1/2}=34 h, TAA-platin: t_{1/2}=58 h, CAA-platin: t_{1/2}=109 h, CIT-platin: t_{1/2}=20 h, DL-MLA-platin: t_{1/2}=13 h, TTA-platin: t_{1/2}=75 h, L-MLA-platin: t_{1/2}=22 h, D-MLA-platin: t_{1/2}=22 h. Therefore, the order of the half-life of 5′-GMP-complex formation activity of the platinum complexes decreases as follows: NTA-platin(168h)>CAA-platin(109h)>TTA-platin(75h)>TAA-platin(58h)>TCA-cisplatin(44h)>TMA-platin(34h)>Cisplatin(22h)=D-MLA-platin(22h)=L-MLA-platin(22h)>CIT-platin(20h)>DL-MLA-platin(13h), with DL-MLA-platin being the most active and NTA-platin the lease active in reacting with 5′-GMP.

### Cytotoxicity of the Platinum Complexes

The cytotoxicity data (Table 2) were obtained by MTT assays. Human cancer cell lines Caco-2 (human epithelial colorectal adenocarcinoma cell) and HepG2 (human liver carcinoma cell) cells were treated with cisplatin at concentration ranging from 2 to 128 μM, and with carboplatin, NTA-platin, TCA-platin, TMA-platin, TAA-platin, CAA-platin, CIT-platin, DL-MLA-platin, TTA-platin, D-MLA-platin and L-MLA-platin at concentrations ranging from 2 to 1,024 μM for 4 days. MTT assays resulted in a dose-dependent inhibition of cell growth and IC₅₀ was obtained via curve fitting (FIGS. 19 and 20).

As seen in Table 2, the new platinum complexes showed much better cytotoxicity (lower IC₅₀) than that of the 2^{nd} generation Pt-based drug carboplatin for both Caco-2 and HepG2 cells, except for NTA-platin and TAA-platin. Moreover, CAA-platin, CIT-platin, DL-MLA-platin, D-MLA-platin and L-MLA-platin exhibited anticancer activity comparable to cisplatin in both Caco-2 and HepG2 cells.

TABLE 3
The IC₅₀ values of the platinum complexes
in two human cancer cell lines

IC₅₀(μM)

| Compounds | Caco-2 | HepG2 |
|---|---|---|
| Cisplatin | 16.72 ± 4.5 | 16.63 ± 4.9 |
| Carboplatin | 209.89 ± 1.6 | 77.42 ± 1.5 |
| NTA-platin | 292.20 ± 60.9 | 555.70 ± 63.2 |
| TCA-platin | 126.00 ± 25.3 | 41.01 ± 4.8 |
| TMA-platin | 61.66 ± 0.8 | 89.13 ± 0.7 |
| TAA-platin | 245.7 ± 0.8 | 315.9 ± 0.6 |
| CAA-platin | 22.2 ± 0.8 | 28.46 ± 0.7 |
| CIT-platin | 13.62 ± 0.8 | 31.14 ± 1.8 |
| TTA-platin | 62.96 ± 0.7 | 51.49 ± 1.9 |
| MLA-platin | 13.57 ± 0.8 | 24.55 ± 1.9 |
| D-MLA-platin | 19.49 ± 0.7 | 15.58 ± 1.5 |
| L-MLA-platin | 22.72 ± 0.7 | 33.08 ± 1.5 |

*IC₅₀ was obtained by MTT assay for 4 days. The value represented as mean ± SD. The experiments were performed in at least 3 independent experiments(n ≥ 3).

### Cytotoxicity of CIT-Platin and CIT-Platin-HER2 Complex Against HER2 Positive and HER2 Negative Human Breast Cancer Cells

Three human breast cancer cells, MCF-7 cell (HER2-non-expressing, HER2−), SKBR-3 and MDA-MB-453 (both are highly HER2-expressing, HER2+) were treated with carboplatin, CIT-platin or CIT-platin-HER2 conjugate at concentrations ranging from 2 to 1,024 μM for 4 days. MTT assay resulted in a dose-dependent inhibition of cell survival.

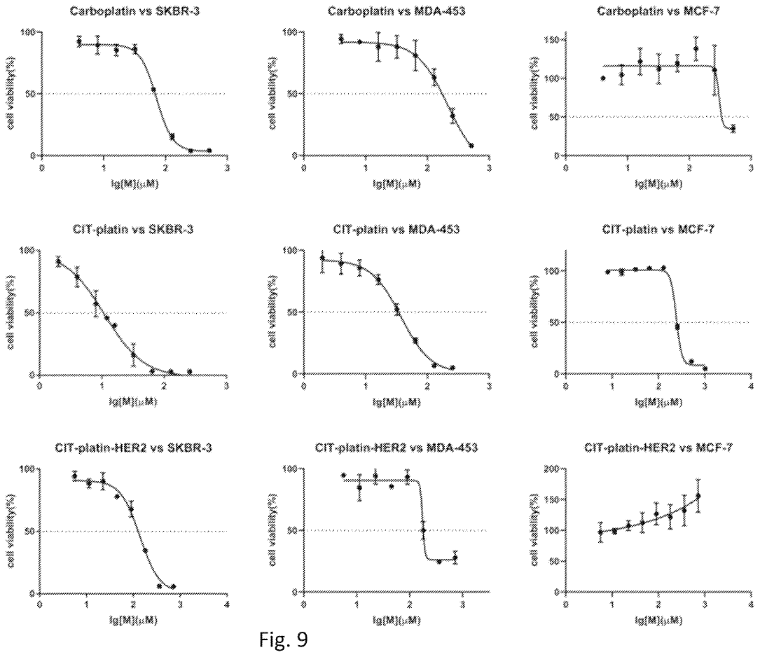

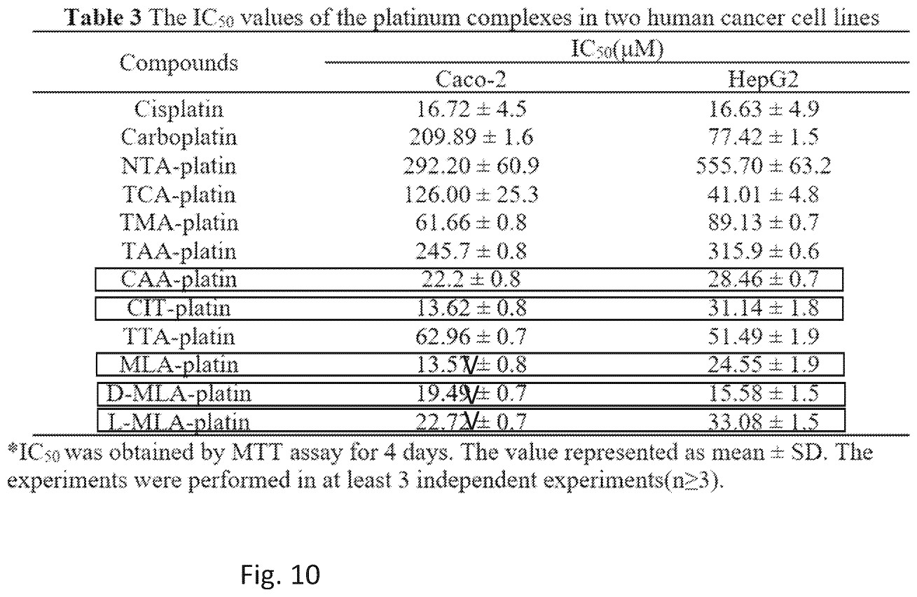

As showed in FIG. 9 and Table. 4, the cytotoxicity of CIT-platin showed increased cytotoxicity compared to carboplatin against all the 3 human breast cancer cell lines, especially the 2 HER2 positive cell lines (6.45 fold decrease in IC₅₀ in SKBR-3 cells and 5.74 fold decrease in MDA-MB-453 cells), suggesting a potential treatment value of CIT-platin in breast cancers.

The cytotoxicity of CIT-platin-HER2 conjugate correlated with the level of HER2 expression in the 3 breast cancer cells, with highly HER2-expressing SKBR-3 and MDA-MB-453 cells being significantly more sensitive than HER2-non-expressing MCF-7 cells. Furthermore, CIT-platin-HER2 conjugate showed no decrease in cellular viability for MCF-7 cells at all concentrations tested including at high concentrations (>1,000 μM), suggesting very low cytotoxicity. This result suggests that the CIT-platin HER2 conjugate may target HER2+ cells (SKBR-3 and MDA-MB-453) but do not target the HER2− MCF-7 cells.

TABLE 4
The IC₅₀ values of CIT-platin and CIT-platin-HER2
conjugates in three human breast cancer cell lines

| | IC₅₀(μM) | | |
|---|---|---|---|
| | SKBR-3 | MDA-MB-453 | MCF-7 |
| Compounds | (HER2+) | (HER2+) | (HER2−) |
| Carboplatin | 71.26 ± 0.61 | 213.2 ± 0.79 | 303.40 ± 33.46 |
| CIT-platin | 11.04 ± 0.76 | 37.13 ± 0.77 | 243.00 ± 0.73 |
| CIT-platin-HER2 | 139.0 ± 0.79 | 175.2 ± 0.81 | — |

*IC₅₀ was obtained by MTT assay for 4 days. The value represented as mean ± SD. The experiments were performed in at least 3 independent experiments(n ≥ 3).

In order to evaluate whether the new platinum complexes induce the death of cancer cells in a similar manner as those of cisplatin or carboplatin, apoptosis assays were performed and monitored under a confocal microscope. HepG2 cells were treated with various concentrations of cisplatin or TCA-platin while SKBR-3 cells were treated with various concentrations of carboplatin or CIT-platin. The cells were incubated at 37 °C, 5% CO₂ in a humidified incubator for 4 days. Cells were treated either with 1 μg/ml hoechst33342(HO) and g/ml propidium iodide (PI) and incubated at 37 °C, 5% CO₂ for 15 min in the dark. After incubating, the cells were examined by a Zeiss LSM 710 fluorescence microscope (Carl Zeiss, Jena, Germany) at respective excitation/emission wavelength of 405/453 nm (HO) and 543/640 nm(PI). Hoechst stains the nuclei of all cells with blue fluorescence, whereas propidium iodide(PI) only penetrates and stains in cells with lost membrane integrity (red fluorescence).

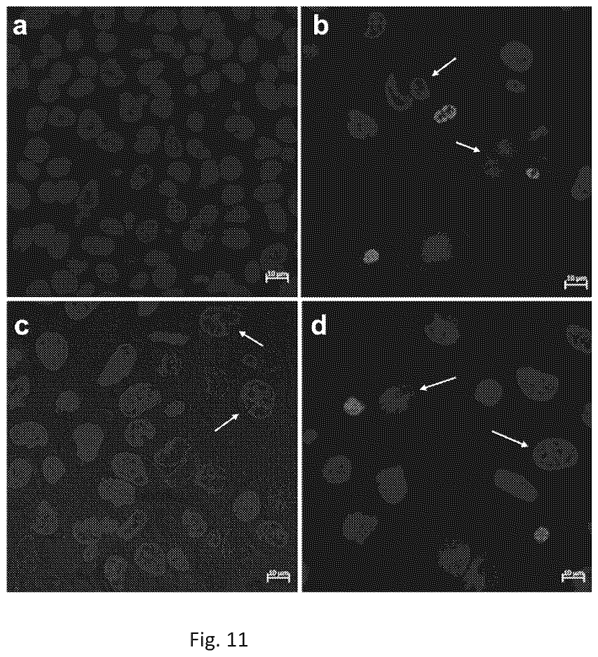

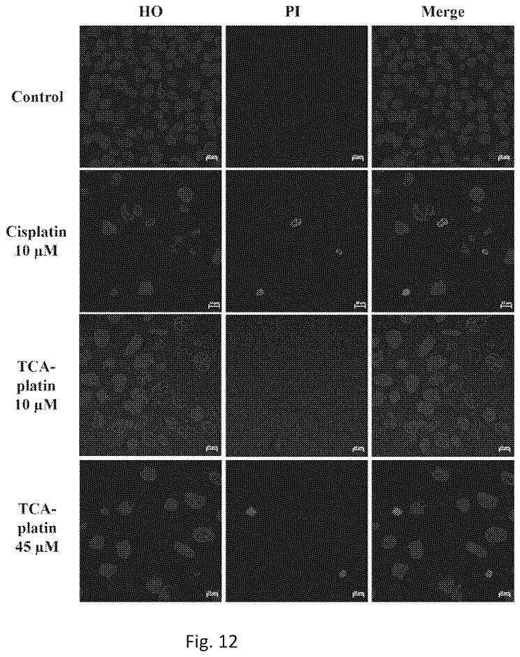

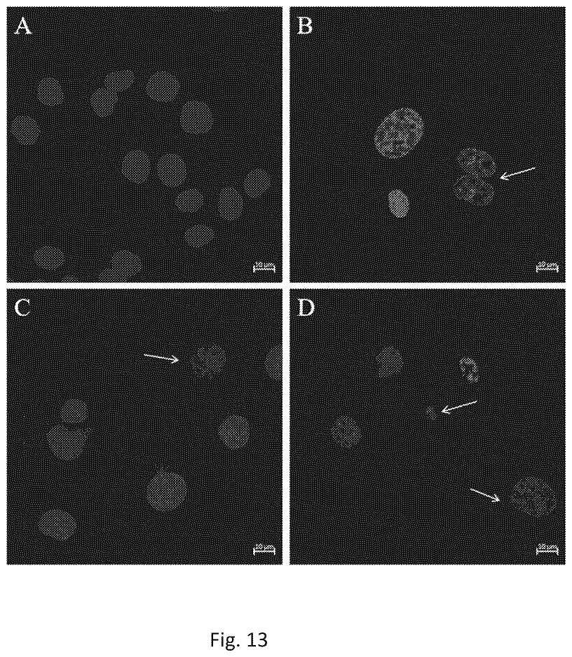

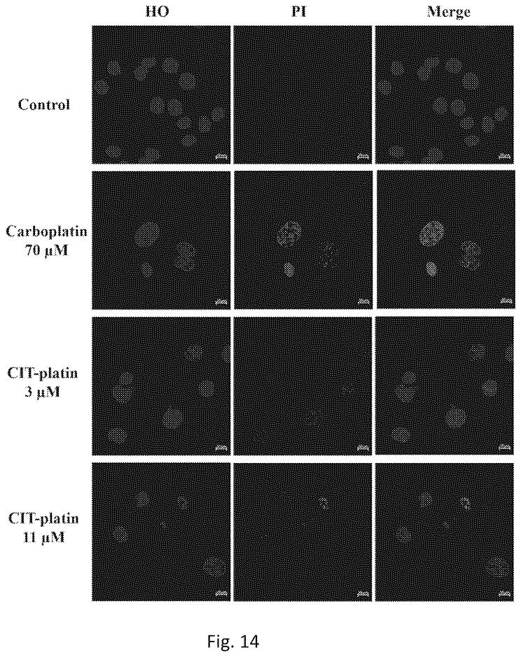

FIGS. 11-14 display the morphological changes of HepG2 and SKBR-3 cells treated with the platinum complexes. A few general conclusions can be made from analysis of the cell imaging data, i.e., 1) the cell numbers decreased dramatically in the presence of each of the platinum complexes; 2) all the platinum complexes tested induce cell nuclei membrane permeability to PI (pink images) and DNA fragmentation similarly, typical biomarkers for apoptosis. It is thus concluded that TCA-platin and CIT-platin induces apoptosis in the cancer cells tested in a similar manner as cisplatin or carboplatin.

TABLE 5
Summary of results

| | | | | Conjugated | | Conjugate | |
|---|---|---|---|---|---|---|---|
| | | Test | Test | to HER2 | Conjugate | test in | Conjugate |
| Compound | Compound | in | in | peptide(Y/ | test in | MDA- | test in |
| name/# | structure | Caco-2 | HepG2 | N) | SKBR-3 | MB-453 | MCF-7 |
| NTA- platin/1 | 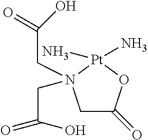 | 292.20 ± 60.9 | 555.70 ± 63.2 | N | | | |
| TCA- platin/2 | 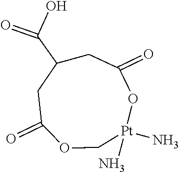 | 126.0 ± 25.3 | 41.01 ± 4.8 | Y | | | |
| TAA- platin/3 | 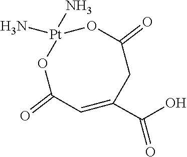 | 245.7 ± 0.8 | 315.9 ± 0.6 | N | | | |
| CAA- platin/4 | 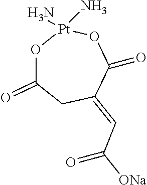 | 22.2 ± 0.8 | 28.46 ± 0.7 | Y | | | |
| TMA- platin/5 | 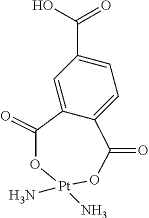 | 61.66 ± 0.8 | 89.13 ± 0.7 | N | | | |
| CIT- platin/6 | 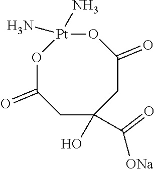 | 13.62 ± 0.8 | 31.14 ± 1.8 | Y | 139.0 ± 0.79 | 175.2 ± 0.81 | No decrease |
| TTA- platin/7 | 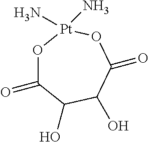 | 62.96 ± 0.7 | 51.49 ± 1.9 | N | | | |
| DL- MLA- platin/8 | 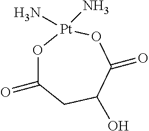 | 13.57 ± 0.8 | 24.55 ± 1.9 | N | | | |
| D-MLA- platin/9 | 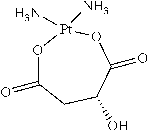 | 19.49 ± 0.7 | 15.58 ± 1.5 | N | | | |
| L-MLA- platin/10 | 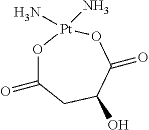 | 22.72 ± 0.7 | 33.08 ± 1.5 | N | | | |
| CIT- DACH- platin/11 | 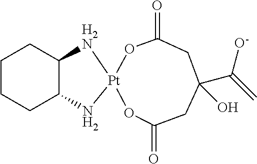 | | | N | | | |

1. A Pt(Tl) complex of Formula (I) or (II): 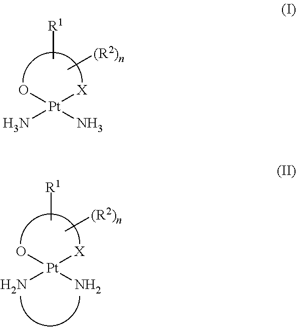
or a pharmaceutically acceptable salt (e.g., carboxylate salts) thereof, wherein:
R¹ is a hydroxyl group (OH), a carboxyl group (COOH) or a carboxyalkyl group (e.g., a C₁-C₄ COOH group);
n is 0, 1 or 2;
R is a hydroxyl group, a carboxyl group (COOH) or a carboxyalkyl group (e.g., a C₁-C₄—COOH group) or two R² groups, together with the atom to which they are attached, can form a double bond (e.g., a double bond optionally substituted with R¹, such as the group ═CHCOOH) or an acyl (CO) group; or
two R² groups, together with the atoms to which they are attached, can form a double bond, a cycloalkyl group or an aryl group, provided that when two R² groups, together with the atoms to which they are attached for an a cycloalkyl group or an aryl group, R¹ can be located on the cycloalkyl group or the aryl group, respectively;
X is O or NR², wherein R² is H or a carboxyalkyl group.

2. The complex of claim 1, wherein the ring formed by: 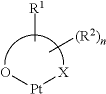
is a five- six-, seven- or eight-membered ring.

3. The complex of claim 1, wherein the ring formed by 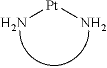
can be a five-, six-, seven- or eight-membered ring that can be fused with another five- or six-membered cycloalkyl or aryl ring.

4. The complex of claim 1, wherein R¹ is OH or COOH.

5. The complex of claim 1, wherein n is 1 and R² is OH or COOH.

6. The complex of claim 1, wherein R¹ is OH or COOH, n is 3, one R² is OH or COOH and the other two R² groups form an acyl group.

7. The complex of claim 6, wherein the group of the formula: 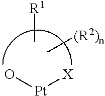
is a group of the formula:

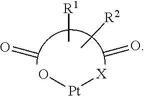

8. The complex of claim 1, wherein the group: 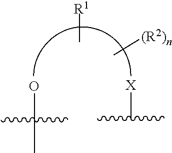
on compounds of the Formula (I) and (II) is a group of the formula: 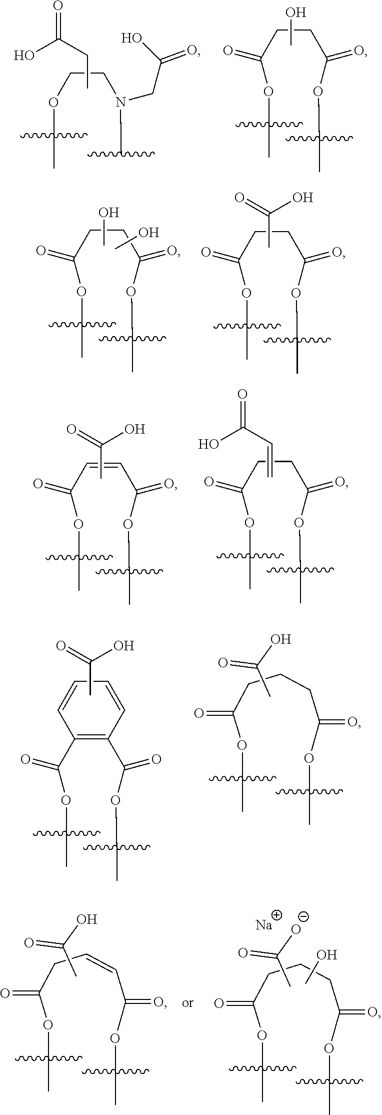
and diastereomers thereof.

9. The complex of claim 1, wherein the group 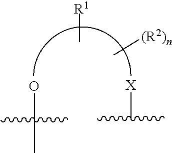
on compounds of the Formula (I) and (II) is a group of the formula: 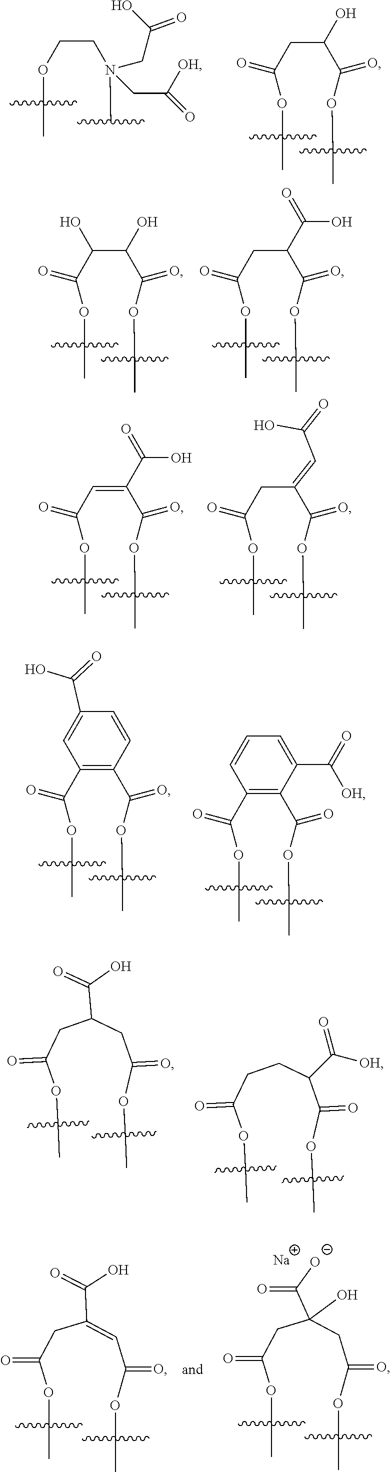
and diastereomers thereof.

10. The complex of claim 8, wherein the group 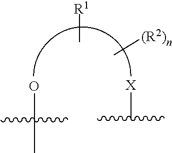
on compounds of the Formula (I) and (II) is a group of the formula:

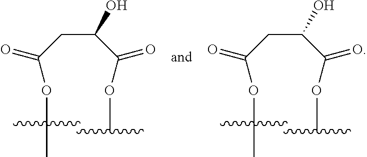 [SMILES: *OC(=O)C[C@@H](O)C(=O)O* ; *OC(=O)C[C@H](O)C(=O)O*]

11. The complex of claim 1, wherein the group 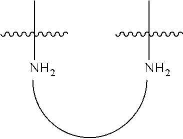
on compounds of the Formula (I) and (II) is a group of the formula:

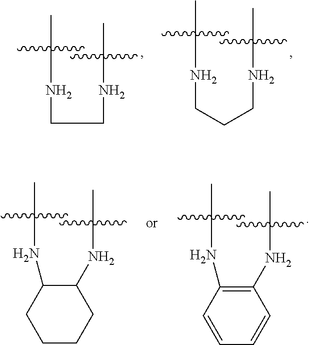 [SMILES: *NCCN* ; *NCCCN* ; *NC1CCCCC1N* ; *Nc1ccccc1N*]

12. The complex of claim 1, wherein the complex is:

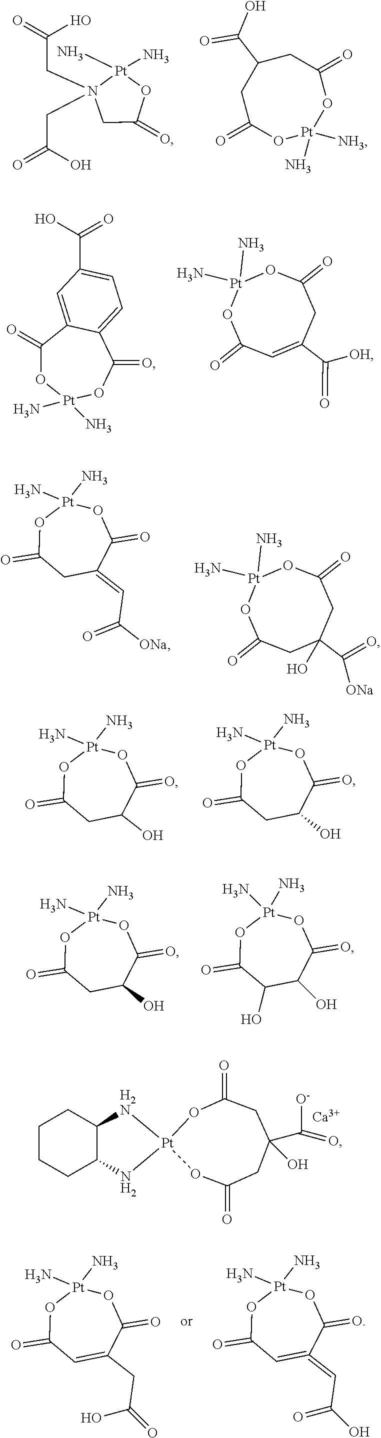

13. (canceled)

14. (canceled)

15. (canceled)

16. (canceled)

17. (canceled)

18. (canceled)

19. (canceled)

20. (canceled)

21. (canceled)

22. (canceled)

23. The complex of claim 1, wherein the complex has a 1.5-, 2-, 3-, 5-, 10-, 15-, 20-, 30-, 50-, 80- or a 100-fold greater water solubility than cisplatin.

24. A method of synthesizing a complex of claim 1, the method comprising:
contacting a starting Pt(II) complex of the general formula (L)₂Pt(amine)₂ with a polycarboxyl compound in the presences of a silver salt, optionally in the presence of a base and/or a salt, to give a Pt(II) complex product of Formula (I) or (II);
wherein:
each L independently represents a leaving group; and
the Pt(II) complex product comprises at least one free carboxyl group.

25. (canceled)

26. The method of claim 24, wherein the amine is NH₃, NH₂R³ (wherein R³ is alkyl), an amine of the formula NH₃-alkyl-NH₃, an amine of the formula NH₃-cycloalkyl-NH₃, or an amine of the formula NH₃-aryl-NH₃.

27. The method of claim 24, wherein the amine is an amine of the formula:

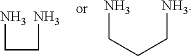

28. The method of claim 24, wherein the amine is an amine of the formula:

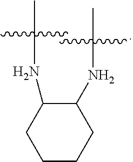 [SMILES: *NC1CCCCC1N*]

29. The method of claim 24, wherein the amine is an amine of the formula:

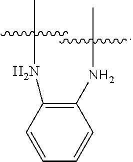 [SMILES: *Nc1ccccc1N*]

30. (canceled)

31. The method of claim 24, wherein the base is NaOH.

32. The method of claim 24, wherein the polycarboxyl compound is a compound of the formula: [chemical structure]
